(19) United States
(12) Patent Application Publication
(10) Pub. No.: US 2011/0189166 A1
(43) Pub. Date: Aug. 4, 2011

(54) METHODS OF TREATING HEMORHEOLOGIC ABNORMALITIES IN MAMMALS

(76) Inventor: John BOUCHER, Silver Spring, MD (US)

(21) Appl. No.: 13/016,984

(22) Filed: Jan. 29, 2011

Related U.S. Application Data
(60) Provisional application No. 61/282,377, filed on Jan. 29, 2010.

(51) Int. Cl. A61K 39/395; C12Q 1/02; G01N 33/53; A61K 38/46; A61K 38/45; A61K 38/44; A61K 38/48; A61K 38/16; A61K 38/06; A61K 38/36; A61P 7/00; A61P 11/00; A61P 31/00

(52) U.S. Cl. 424/131.1; 435/29; 435/7.24; 424/94.6; 424/94.5; 424/94.4; 424/94.64; 424/172.1; 514/13.5; 514/13.7; 424/152.1; 424/173.1

(57) ABSTRACT

Methods of treating hemorheologic abnormalities in mammals are provided, as well as methods of evaluating circulatory flow mechanics by analyzing hemorheologic determinants or hemorheologic abnormalities in the blood.

### RELATED APPLICATIONS

This application claims priority from U.S. Provisional Patent Application No. 61/282,377, filed Jan. 29, 2010, which is hereby incorporated by reference in its entirety.

### FIELD OF THE INVENTION

This invention relates generally to hemorheology, the study of circulatory flow mechanics, and, more particularly, to methods of treating hemorheologic abnormalities in mammals, as well as methods of evaluating circulatory flow mechanics by analyzing hemorheologic determinants or abnormalities in the blood.

### BACKGROUND

Hemorheology, the science of circulatory flow mechanics, provides an important link between abnormal blood physiologic function and disease or premature fatigue or reduced performance following exposure to a non-disease related stress, such as exercise. Previously, it has been reported that exercise induces an increase in crenated or spiculated red blood cells (echinocytes) in horses. U.S. Pat. No. 4,383,997. It was also found that certain compounds that inhibit the influx of extracellular calcium or that increase intracellular ATP, such as pentoxyfylline, inhibit the increase of crenated red blood cells following exercise. U.S. Pat. No. 4,383,997. At the time, however, the reason for the appearance of the crenated or speculated red cells during exercise was unknown. Furthermore, the far-reaching effects of crenated or speculated red blood cells in other pathophysiologies and diseases were not understood. Similarly, the complex interaction of multiple pathways involving other hemorheologic determinants had not been discovered. Thus, the hemorheologic science based, in part, on blood hyperviscosity syndromes, opens an opportunity for the discovery of new therapies and diagnostic modalities for managing premature fatigue, reduced performance, or a variety of diseases in both animals and humans.

### SUMMARY

Circulatory flow mechanics comprises a complex interaction of multiple pathways that can act individually, or in concert, to give rise to a hemorheologic abnormality, namely, an increase in a blood viscosity determinant, an increase in phosphatidlyserine exposure, or an increase in the expression of adhesion molecules on the surface of blood or endothelial cells. Often times, when left untreated, the hemorheologic abnormality can give rise to a pulmonary or systemic disease. Alternatively, the hemorheologic abnormality occurs in the blood of a healthy subject following a non-disease related stress, including, but not limited to, exercise, exposure to a hot and humid environment, skin burn, exposure to high altitude, underwater diving, hypoxia (tissue oxygen deprivation), surgery, blood storage lesions, or space travel. Strategically targeting one or more of these pathways that influence circulatory flow mechanics, thus, provides an opportunity to treat a hemorheologic abnormality, which may be associated with either a disease or a non-disease related stress.

Accordingly, the present invention is directed to methods of treating a hemorheologic abnormality in a mammal comprising administering to said mammal an effective amount of a hemorheologically-active compound. In certain embodiments, the method comprises the additional steps of measuring one or more hemorheologic determinants in the blood of said mammal before and after administering the hemorheologically-active compound and analyzing the measurements to assess the effectiveness of administering the compound.

Hemorheologic abnormalities result in reduced blood flow, increased resistance to blood flow, and tissue oxygen deficit in the systemic system or hypertension in the pulmonary system leading to systemic or pulmonary pathology or pathophysiology or premature fatigue or reduced performance. Thus, in certain embodiments of the methods described herein, the hemorheologic abnormality is associated with a pulmonary disease or a systemic disease. In other embodiments, the hemorheologic abnormality is associated with a non-disease related stress, including, but not limited to, exercise, exposure to a hot and humid environment, skin burn, exposure to high altitude, underwater diving, hypoxia, surgery, blood storage lesions, or space travel.

The present invention is also directed to methods of evaluating hemorheologic determinants or hemorheologic abnormalities in the blood to differentiate normal blood flow mechanics from dysfunction. These methods enable not only the detection of abnormalities in blood flow mechanics, but also early detection of disease or pathophysiology. They also permit one to asses the effectiveness of a compound in treating a hemorheologic abnormality, whether it is associated with a pulmonary or systemic disease or a non-disease related stress.

### DETAILED DESCRIPTION

Reference will now be made in detail to the exemplary embodiments of the invention. These embodiments are described in sufficient detail to enable those skilled in the art to practice the invention and it is to be understood that other embodiments may be utilized and that changes may be made without departing from the scope of the invention. The following description is, therefore, merely exemplary.

The term “administering” refers to the administration of a hemorheologically-active compound, either prophylactically or therapeutically. When provided prophylactically, a hemorheologieally-active compound may be administered in advance of exposure to a stress or the onset of a symptom associated with a hemorheologic abnormality or a disease associated with a hemorheologic abnormality. When provided therapeutically, a hemorheologically-active compound may be administered at (or after) exposure to a stress or the onset of a symptom associated with a hemorheologic abnormality or a disease associated with a hemorheologic abnormality.

As used herein, the term “effective amount” or “therapeutically-effective amount” refers to an amount that will result in treatment of the hemorheologic abnormality and may readily be determined by one of ordinary skill in the art. An effective amount of a hemorheologically-active compound is typically an amount such that when it is administered in a physiologically tolerable excipient composition, it is sufficient to achieve an effective concentration in the circulatory system or an effective local concentration in a target tissue. The activity contemplated by the present methods includes both therapeutic and/or prophylactic treatment, as appropriate. The specific dose of a compound administered to obtain therapeutic and/or prophylactic effects will be determined by the particular circumstances surrounding the case, including, for example, the compound administered, the route of administration, and the condition being treated.

The term “hemorheologic determinant” refers to a factor that influences circulatory flow mechanics and includes, but is not limited to, a blood viscosity determinant, phosphatidlyserine exposure, and expression of adhesion molecules on the surface of blood or endothelial cells.

The term “hemorheologic abnormality” refers to an abnormal level of a hemorheologic determinant in a subject. More specifically, the term refers to an increase in a blood viscosity determinant, an increase in phosphatidlyserine exposure, or an increase in the expression of adhesion molecules on the surface of blood or endothelial cells, as compared to a normal level of said hemorheologic determinant in said subject. If left untreated, a hemorheologic abnormality may impede blood flow and cause pathophysiology or disease.

The term “blood viscosity determinant” is a conventional term used by hemorheologists to refer to factors which, if altered (individually or in combination), can increase blood viscosity to abnormal levels. The most common blood viscosity determinants include red blood cell concentration, red blood cell aggregation, red blood cell rigidity (resulting in reduced deformability), plasma viscosity, and abnormal red blood cell shape (e.g., formation of echinocytes, stomatocytes, or elliptocytes).

The term “hemorheologically-active compound” refers to a compound that improves blood flow by reducing an abnormal level of a hemorheological determinant.

The term “phosphatidylserine exposure” refers to phosphatidylserine exposed on the outer surface of blood cell plasma membranes.

The term “adhesion molecules” refers to proteins located on the surface of cells and involved in mediating binding or adhesion between one or more cells. Examples of adhesion molecules include, but are not limited to, a selectin protein (e.g., P-selectin, L-selectin, or E-selectin), a fibrinogen protein, an immunoglobulin (e.g., IgG, IgA, IgM), an I-CAM protein, a V-CAM protein, an integrin protein (e.g., VLA-4, LFA-1, macrophage-1 antigen), an addressin protein, and a cadherin protein (e.g., E-cadherin).

The term “pulmonary disease” refers to a pathology or a pathophysiology caused by a flow impediment or blockade of the pulmonary circulation and includes, but is not limited to, pulmonary hypertension (arterial or venous), intrapulmonary right-left shunting, hypoxemia, exercise-induced pulmonary hemorrhage, fibrosis, hemosiderosis, vascular remodeling (hyperplasia), and partial or complete veno-occlusion (arterial or venous).

The term “systemic disease” refers to a pathology or a pathophysiology caused by a flow impediment or blockade of the systemic circulation and includes, but is not limited to, myopathy (muscle disease), laminitis, navicular disease, gastric ulcers, subepiglottic ulcers, bone demineralization, musculoskeletal failure, bone cysts (aseptic necrosis), laryngeal hemiplegia, cardiac arrhythmias, myocardial fibrosis, degenerative joint disease, degenerative myeleoncephalopathy, osteochondrosis, liver disease, kidney disease, vascular wall damage, vascular remodeling (hyperplasia), myocardial infarction, stroke, sickle cell disease, diabetes, β-thalassemia, cancer, antiphospholipid syndrome, endotoxemia, uremia, malaria, sepsis, hepatitis, ischemia-reperfusion, blood storage lesions, Alzheimer's disease, Duchenne muscular dystrophy, eclampsia, glucose-6-phosphate dehydrogenase deficiency, and Wilson's disease.

The term “non-disease related stress” refers to a stress experienced by an otherwise healthy subject and includes, but is not limited to, exercise, exposure to hot or humid environment, skin burn, exposure to high altitude, underwater diving, hypoxia, surgery, or space travel.

The terms “treatment” or “treating” and the like refer to any treatment of any disease or condition in a mammal and includes reducing or preventing a disease, condition, or symptom of a disease or condition, e.g., arresting its development and/or delaying its onset or manifestation in the patient or relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired.

The terms “healthy” or “healthy subject” refer to a subject having no diagnosed or discernible symptoms of a disease.

The term “pharmaceutically acceptable carrier” or “pharmaceutically acceptable excipient” means solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art.

As used in this specification and the appended claims, the singular form “a,” “an” and “the” include plural referents unless the context dictates otherwise.

### Hemorheology

Hemorheology, the study of circulatory flow mechanics, earns respect as the only science that brings unity to all body systems; i.e., the blood-vessel organ is the only organ of the body that directly interacts with, and affects, all other body systems. The mechanisms and magnitude of hemorheopathy (abnormal blood flow mechanics) observed in exercising horses is uniquely different from, and greater than, that of any other species. Surprisingly, healthy exercising horses generate increases from resting (control) values in all hemorheologic determinants, including phosphatidylserine exposure. These increases can lead to both pulmonary and systemic diseases. Other species, including humans, can also generate hemorheopathy following exposure to a non-disease related stress (e.g., intense exercise or heat), but not to the extent of that developed in healthy horses. However, severe hemorheopathy occurs in other species, including humans, during certain diseases on the order of magnitude similar to that generated by healthy horses during any stressful event, including exercise. To understand how hyperviscous blood could impede blood flow and cause pathophysiology or disease, requires some knowledge of normal flow mechanics as well as understanding the determinants of blood viscosity and other theological abnormalities.

Blood flow mechanics involves the dynamic forces and resistances bearing on plasma and blood cells as it traverses circulatory segments of various sizes (arteries, arterioles, capillaries, venules, veins). The main forces are generated by the heart's pumping forces, the velocity of flow, the size of vessels, the interactions of plasma and blood cells, blood cell-cell interactions, and the interaction of blood cells and the vessel wall. This implies that blood exhibits a variable viscosity depending upon how rapidly it is forced to flow under shear, and depending upon the health status of blood cells.

### Viscosity

Viscosity is a measure of the ease with which flow occurs in a liquid. The greater the viscosity of a liquid, the slower it will flow. More precisely, viscosity is a measure of the internal friction within a flowing liquid. For example, oil is more viscous than water because oil has greater friction between flowing adjacent layers than does water. Blood viscosity relates to the internal resistance of blood to shear forces. For example, an abnormality of blood cells, such as echinocytes (horses & humans) or sickle cells (humans), or large, intensely bound aggregates of blood cells, increases the internal friction of flowing blood by disrupting laminar flow streamlines and, accordingly, increases blood viscosity. Increases in the cellular concentration of blood also increases viscosity. Viscosity is a function of the velocity gradient between adjacent layers of flowing blood and the molecular friction of blood, and is an indicator of blood fluidity. The greater the fluidity (decreased viscosity; decreased resistance), the easier blood flows.

Viscosity is defined by the proportionality of shear rate to shear stress. For blood, a non-Newtonian fluid, the relation of stress to strain rate is nonlinear.

Viscosity(η)=Shear stress(τ)/Shear rate(γ)

Operationally, this relationship can be more simply defined: Viscosity is proportional to force of flow/velocity of flow.

### Blood Flow and Resistance to Flow

Blood flow (Q; cardiac output) to tissues is determined by the ratio between the driving pressure (ΔP, arterial-venous pressure difference) and the flow resistance (R).

Q=ΔP/R

Resistance is a common-sense quantitative measure of how much difficulty exists in driving the flow through a given section of the vascular bed (AP/Q), i.e., it measures the extent to which the system resists flow. The resistance through a vascular network is dependent on both the geometric features of the vessels (constriction or dilation) and the flow properties of the blood. Thus, resistance may be expressed as a product of vascular hindrance (Z) and blood viscosity (nB).

R=ΔP/Q=Z×ηB

Note that Z and ηB are not added to each other but rather are multiplied to give the resistance to flow. A small increase in blood viscosity can therefore amplify the effect of elevated vasoconstriction (increased Z) in raising the flow resistance, or vice versa.

### Hemorheological Determinants

A hemorheologic abnormality refers to an abnormal level of a hemorheologic determinant, namely a blood viscosity determinant, phosphatidylserine exposure, and adhesion molecules. Of all the factors influencing hemorheological abnormalities, blood viscosity determinants are probably the most important and have received the most attention throughout the development of the hemorheologic science. Exercising horses generate increased values in all blood viscosity determinants far beyond those of a healthy human. Indeed, the blood of exercising horses represents the least favorable mechanical flow properties (hemorheology) of any species during exercise or disease. Each hemorheologic determinant is examined in further detail below.

1. Blood Viscosity Determinant

Blood viscosity determinants are factors which, if altered (individually or in combination), can increase blood viscosity to abnormal levels. The most common blood viscosity determinants include red blood cell concentration, red blood cell aggregation, red blood cell rigidity (resulting in reduced deformability), plasma viscosity, and abnormal red blood cell shape (e.g., formation of echinocytes, stomatocytes, or elliptocytes).

a. Red Blood Concentration

Hematocrit (Hct) is the prime determinant of blood viscosity, i.e., the more cells, the greater the viscosity. The Hct increase of horses during exercise is unique. Unlike that of exercising humans, the Hct of exercising horses increases to polycythemic levels. A Hct percent change from rest to maximal exercise of 60-65% is typical of a high performance horse. This phenomenon is due to splenic contraction. Unlike the human spleen, the equine spleen, an adrenergically-controlled organ, sequesters nearly half the total erythrocyte mass, and upon exercise it mobilizes the sequestered erythrocytes into the circulation. This explains the difference between the horse and human hematocrit induced by exercise. The horse's Hct difference between rest and maximal exercise represents a four-fold increase in blood viscosity.

b. Red Blood Cell Aggregation

Two distinctly different erythrocyte rheologic behaviors manifest in two phenomena, aggregation or deformation, depending upon the shear rate of flowing blood. Those phenomena (aggregation and deformation) account, in part, for the variability of blood viscosity. Blood is a complex fluid that has a potential for either high or low viscosity depending upon the shear rate. Blood changes its configuration as it flows in the circulation; particles in the central axis of vessels travel fastest. Shear rate (proportional to velocity of flow) differs throughout the circulation depending upon the vessel diameter and blood flow velocity. Shear rate is the gradient of velocities of adjacent fluid layers within a vessel and is highest in the arterioles and capillaries and lowest in the venules.

In streamlined (laminar) flow, adjacent layers of liquid move in parallel, with the fastest at the center and the slowest at the wall of the vessel. The relative flow rate between adjacent layers is called the “shear rate” (defined as a velocity gradient). The shear rate (s⁻¹, a dimensionless variable) in turn depends upon the vessel diameter and blood flow velocity. Almost all of the friction takes place between adjacent layers within the flowing liquid; this property is called “shearing within the liquid. The shear rate-dependent viscosity-altering influence of erythrocytes gives blood a non-Newtonian behavior, i.e., blood viscosity differs with shear rate. The non-Newtonian feature of blood distinguishes it from Newtonian fluids such as water or oils, the viscosities of which are not altered by changes in shear rate.

A wide range of shear rates exists throughout different parts of the circulation. Shear rates above 100 sec⁻¹ occur in all large blood vessels; it increases through the branching of small arteries and arterioles and reaches a maximum in the capillaries. The lowest shear rates are noted in the venules and small veins, where a “near” flow stasis occurs. In these vessels, erythrocyte aggregation can occur. A 50-fold change in shear rates exists within the circulation. For that reason, blood viscosity must be measured over a wide range of viscometer shear rates to characterize fully the flow behavior of blood.

Low shear rate (around 10 s⁻¹ and less) induces erythrocytes to aggregate and generate a high blood viscosity. Low shearing forces allow the cells to interact (unlike dispersed cells under high shearing forces). The high viscosity of whole blood at low shear rates results, in part, from erythrocyte aggregates formed by the bridging of the cell surfaces by plasma fibrinogen and globulins. With increasing shear rates, aggregates break up, cells become dispersed, and a reduction in viscosity occurs.

Plasma protein macromolecules such as fibrinogen and the immunoglobulins (e.g., IgA, IgG, and IgM) promote RBC-RBC binding when the cell's environment contains little or no shearing forces (low velocity) as in venules. Blood becomes “sludge-like” and the viscosity value rises to a very high level relative to the value at high shear rate where erythrocytes are dispersed, deform (elongate), and “tank tread” (described later). First rouleaux form, followed by aggregation, which is a rouleaux-rouleaux three-dimensional structure. The magnitude (size) and intensity (strength) of the aggregation determines aggregation pathology. An intense (pathological) aggregation takes greater shear forces to disaggregate the cells. Aggregations of larger size (more cells) disturb flow laminations to a greater extent than small ones and cause a pathological hyperviscous state of the blood. Another form of erythrocyte aggregation, clumping (agglutination-like), most likely has an immunogenic origin; because of its much greater binding intensity than the rouleaux-rouleaux aggregation, agglutination aggregates take higher shear rates to disperse and are pathological. Intensely bound erythrocyte aggregates can become large and rigid, obstruct microcirculatory vessels, and lead to systemic hypoxia or pulmonary hypertension. Dintenfass reported aggregations of up to 100,000 red blood cells in human patients with cardiovascular disease (Dintenfass, L., Biorheology, 25:65-76, 1988). Aggregation tendency is dependent upon: (1) red blood cell membrane abnormality, or (2) increased plasma adhesion molecules, or (3) slow flow rate (low shear rate).

c. Red Blood Cell Rigidity

Red blood cell deformation occurs at high shear rate (above 100 s⁻¹) where the flowing blood's laminar shear stress acts on the whole cell causing it to elongate; adjacent laminae move at different velocities (fastest near the vessel axis) and causes the membrane to rotate (“tank treading”) and starts the intracellular contents swirling. The erythrocyte's tank treading and swirling cytoplasm participate in the flowing blood as a water droplet rather than as a rigid particle in suspension. Under this high shear rate condition, blood becomes more fluid, and the viscosity value becomes progressively lower as the shear rate increases due to erythrocyte deformability. Blood viscosity levels then reach levels that approach those of plasma.

The red blood cell mechanical property, deformability, is important for effective blood circulation. The biconcave shape of discocytes provides the cell with a large surface area (SA) in relation to its cellular volume (V). The large SA to V ratio allows the membrane to be deformable (flexible; bendable) in response to blood flow shearing forces. Therefore, deformability lowers blood viscosity and provides the erythrocyte with a property that allows it to pass through channels much smaller than its own diameter. On the other hand, increased rigidity of the red blood cell reduces the ability of the cell to bend its membrane, causing the red blood cell to lose its biconcave shape and preventing the red blood cell from deforming. For this reason, rigid red blood cells have difficulty passing through small microcirculatory vessels with diameters less than the rigid red blood cell. Therefore, large numbers of rigid red blood cells cause oxygen deficit to affected tissue and contribute to disease.

d. Plasma Viscosity

Plasma viscosity is slightly higher than that of water. The only constituents of plasma that have an effect on blood viscosity are fibrinogen and macromolecular globulins (e.g., IgG, IgA, and IgM). Albumin has no effect on blood viscosity. The reason for the difference between the various proteins is based upon their molecular weight and their structural geometry. The molecular weight of the albumin molecule is low, whereas fibrinogen and the globulins are high molecular weight proteins with an oblong geometry. Therefore, when present in high concentrations, the latter proteins increase the internal flow friction of plasma, causing an elevation in viscosity. Increased plasma viscosity in the horse is associated with an elevated turnover of fibrinogen, the mechanism of which is unknown.

e. Red Blood Cell Shape Change

Boucher has previously reported increased numbers of abnormally-shaped red blood cells, called echinocytes, in exercising horses (Boucher, J. H, Exercise-induced echinocytosis. Jones, W. E. (ed.), Equine Sports Medicine, Chapter 4. Philadelphia: Lea & Febiger; 1988). Echinocytes are membrane altered erythrocytes with spicules (i.e., blebs, projections) over the cell surface and tend toward a spherical shape. Echinocyte shapes develop in four stages (Bessis classification) with increased severity of the biochemical changes in the cell. Normal erythrocytes (discocytes) arc biconcave cells without deviation in shape. Atypical, bizarre shapes are typical of intermediate stage echinocytes in horse blood. Greater than 50% of the circulating erythrocytes are echinocytes in the blood of exercising horses, whereas, the numbers of echinocytes in the blood of resting horses is much lower, around 15 percent. Considering the polycythemia developed during exercise, the “absolute” increase of echinocytes could be about 125% change from that at rest.

Echinocytes are rigid cells due to a decreased surface area-to-volume ratio, which occurs because the tips of spicules “bud off” and reduce their surface area without a change in cellular volume. Their rigidity causes an impeded blood flow and reduces tissue oxygenation in much the same way as do small populations of rigid (human) sickle cells, i.e., by remaining at the precapillary site for longer periods than cells with normal deformability they would disproportionately occupy most of the capillaries and reduce flow to a much greater extent than would be expected on the basis of their percent of the total red cell population.

Echinocytes have also been observed in various human diseases and environments, including, but not limited to, 1) sickle cell disease (Mohandas, N. et al., Association between morphologic distortion of sickle cells and deoxygenation induced cation permeability increase. Blood 68:450-454, 1986), 2) burns (Harlan, W. R., et al. Echinocytes and acquired deficiency of plasma lipoproteins in burned patients. Arch. Intern. Med. 136:71, 1976), 3) liver disease (Owen, J. S. et al., Erythrocyte echinocytosis in Liver Disease: Role of abnormal plasma high density lipoproteins. J. Clin. Invest. 76:2275-2285, 1985), 4) renal disease (Udden, M. M. et al., Decreased deformability of erythrocytes and increased intracellular calcium in patients with chronic renal failure. Clin. Hemorheol. 4:473-481, 1984), 5) eclampsia (Cunningham, F. G. et al., Erythrocyte morphology in women with severe preeclampsia and eclampsia: Preliminary observations with scanning electron microscopy. Am. J. Obstet. Gynecol. 153:358-363, 1985), 6) Duchenne muscular dystrophy, 7) extracorporeal circulation during surgery (Karnak T. et al., Erythrocyte crenation induced by free fatty acids in patients undergoing extracorporeal circulation. Lancet 2(8563):818-821, 1987), 8) deep-sea diving (Carlyle, R. G. et al., Abnormal red cells in blood of men subjected to simulated dives. Lancet 1:1114-1116, 1979), 9) exposure to high altitude (Rowles, P. M. and E. S. Williams, Abnormal red cell morphology in venous blood of men climbing at high altitude. Brit. Med. J. 286:1396, 1983), 10) exercise (Selby, G. B. et al., Athlete's echinocytes: New cause of exertional hemolysis. Blood 70:56a, 1987; Connes, P. and J. H. Boucher, Echinocytosis in athletes with exercise-induced hypoxemia, Clinical Hemorheology and Microcirculation, 44:107-114, 2010), 11) space flight (Kimzey, S. L., Hematology and Immunology Studies. Chap. 28, In: Johnson, R. S. and L. F. Dietlein (eds.). Biomedical Results from Skylab. NASA SP-377, Washington, D.C., 1977, pp. 249-282), 12) outdated blood for transfusion (Laczko, J. et al., Discocyte—echinocyte reversibility in blood stored in CPD over a period of 56 days. Transfusion. 19:379-88, 1979), 13) enzymopathies of red blood cell glycolysis, 14) hypophosphatemia, and 15) blackfoot disease. Notwithstanding the observation of echinocytes in various diseases and environments, there have been no published reports of selecting compounds to treat echinocytosis.

The shape change from discocyte to echinocyte occurs when a membrane perturbation collapses the normal phospholipid asymmetric distribution and causes phospholipid randomization. Phosphatidylserine, a molecule normally held unexposed in the membrane inner bilayer leaflet becomes exposed on the membrane outer surface (a condition discussed in detail later).

Another example of an abnormally shaped red blood cell is the stomatocyte. Stomatocytes are characterized by a pale, elongated, mouth-like area in the center of the cell. The change in shape is usually associated with a decrease in the ratio of surface area-to-volume that can be induced either by a reduction in surface area or an increase in red cell volume. The decreased ratio of surface area to volume often causes the stomatocytes to become trapped in the microvasculature of the spleen and other organs of the monocyte/macrophage system, producing varying degrees of hemolysis.

Stomatocytosis is associated with both congenital and acquired diseases. The most common congenital form is hereditary stomatocytosis, a genetic disorder that comprises a variety of different syndromes. Healthy subjects normally have less than 3% of stomatocytes circulating in their blood, while a much higher percentage (up to 40 to 60%) is observed in patients with hereditary stomatocytosis (Kanzaki et al., Br J Haematol 1992; 82:133) and 10 percent or more in some patients with acquired disease such as alcoholism (Wislöff et al., Scand J Haematol 1979; 23:43).

Yet another example of an abnormally shaped red blood cell is the elliptocyte. Elliptocytes, also called ovalocytes, appear oval or elongated in shape and are rich in hemoglobin. These abnormally shaped red blood cells are observed in hereditary disorders, such as hereditary elliptocytosis, or in acquired disorders, such as iron deficiency anemia, infectious anemias, thalassemia, and in newborn babies.

2. Phosphatidylserine Exposure

Normal biconcave erythrocytes transform into echinocytes after a membrane perturbation induces phospholipid redistribution (Deuticke, 1968; Sheetz & Singer, 1974) and causes phosphatidylserine (“PS”) expression on the cell's surface (Zwaal & Schroit, 1997). PS is now known as a indispensible component for maximizing the coagulation process (Zwaal et al, 1998).

Membranes of normal erythrocytes contain four major phospholipid classes distributed asymmetrically between the lipid bilayers (Zwaal et al, 1975). Phosphatidylcholine, (PC) and sphingomyelin, (SM) localize mainly in the outer bilayer. Phosphatidylethanolamine (PE), however, resides mainly in the inner bilayer, while phosphatidylserine (PS) resides there exclusively (Verkleij et al, 1973). PE and PS are negatively charged aminophospholipids unlike the neutralcholine-containing phospholipids, PC and SM. This selective asymmetric localization dictates that the biomembranes are assembled and maintained by specific mechanisms that regulate transbilayer lipid sidedness. Two ATP-dependent active transporters maintain the phospholipids in a state of dynamic asymmetric equilibrium between the bilayers (Connor et al, 1992). One, termed flippase, rapidly transports PE and PS from the outer to inner bilayer. The other, termed floppase, slowly transports all phospholipids non-specifically from the inner to outer bilayer. The two enzymes work synchronously and cooperatively to maintain the membrane phospholipids in their asymmetric locations (Zwaal & Schroit, 1997). The rapid regulation of aminophospholipids (PE and PS) by flippase affects net equilibrium distribution by placing most of the PE and all of the PS in the inner bilayer (Seigneuret & Devaux, 1984), However, inactivation of the energy-driven enzymes (flippase and floppase) by metabolic degradation or oxidative stress 1) disrupts the normal membrane asymmetry; 2) generates a PS-exposed procoagulant cell, and 3) induces a morphologic change of normal erythrocytes into echinocytes (Kamp et al, 2001).

The normal membrane phospholipid asymmetry collapses under conditions of red cell metabolic degradation, oxidative stress, or acidification; the energy-driven enzymes (flippase and floppase) along with the calcium pump (Ca⁺⁺ ATPase) inactivate. As a result of the calcium pump shutdown, intracellular calcium accumulates and, at μM levels, calcium becomes the major regulator of pathological phospholipid distribution between the bilayers (Williamson et al, 1992). Increased intracellular calcium activates calcium-sensitive potassium channels (Gardos channel) in the membranes of red blood cells, resulting in cell shrinkage due to loss of potassium and chloride ions and water. Excess intracellular calcium further inactivates flippase (Bitbol et al, 1987) preventing the rapid outer-to-inner membrane bilayer transport of aminophospholipids (PS and PE) and translocase, a rapid transporter of aminophospholipids from the outer lipid monolayer to the inner monolayer, thereby inhibiting the transport of PS to the inner membrane of the phospholipid bilayer. Calcium excess also activates yet another enzyme termed scramblase (Comfurius, et al, 1990). Activated scramblase transports all phospholipids (non-specifically) between bilayers very rapidly; both the choline (PC and SM) and amino (PE and PS) phospholipids migrate randomly between bilayers. Phospholipid asymmetry collapses, and the normally cryptic PS becomes exposed on the membrane surface. Increased intracellular calcium also activates calpain, a cysteine proteinase that hydrolyzes red blood cell proteins, causing membrane vesiculation. In addition, intracellular acidification also accelerates transbilayer random migration of phospholipids and induces PS exposure on erythrocytes (Libera et al, 1997; Stout et al, 1997). Even a small percent of PS exposure can transform discocytes into echinocytes (Seigneuret & Devaux, 1984; Daleke & Huestis, 1989; Geldwerth et al, 1993; Lin et al., 1994; Kamp et al., 2001).

PS exposure on echinocytes promotes major pathologic conditions that can impede or inhibit microcirculatory blood flow. For example, exposed PS promotes coagulation and thrombosis by providing a catalytic surface for the assembly and stimulation of coagulation protein complexes (prothrombinase and tenase) leading to a dramatic increase in the formation rate of thrombin (Zwaai and Schroit, 1997). No other phospholipid stimulates coagulation proteins as efficiently as PS (Zwaai et al, 1998; Andree et al, 1995; Dachary-Prigent et al, 1996). Only 10-15 mol % of PS on the cell surface (Gerards et al, 1990) results in more than a million-fold enhancement of coagulation cascade enzymatic conversion efficiency (Zwaal et al, Mol Cell Biochem 91:23-31, 1989) enabling microthromboembolic generation. In addition, exposed PS binds to thrombospondin causing erythrocyte-endothelium adhesion thereby increasing the resistance to blood flow in venules where flow shear rates are lowest (Closse et al, 1999; Manodori, et al., 2000). Further, coincident with PS exposure, calpain activated by calcium (Pasquet et al, 1996) facilitates membrane spiculation and release of PS-expressing procoagulant microparticles (fragmented membrane) from echinocytes (Setty et al, 2000); the exposed PS on these microparticles adds to the coagulation potential of blood.

Kamp et al. (2001) reported that changes in the extent of scrambling (randomization) of membrane phospholipids correlates with changes in the extent of echinocytosis and membrane microvesiculation (microparticles). Their work emphasizes that PS exposure precedes a cellular shape change (echinocytosis). In other words, echinocytes are cells with PS exposure. Accordingly, echinocytosis predicts the presence of externalized PS and, therefore, becomes a marker for potential microcirculatory hemorheopathy. Reports also confirm that exposed PS on echinocytes and associated microparticles in humans impede microcirculatory blood flow by rheologic mechanisms associated with coagulation. More specifically, human diseases in which PS exposure has been reported include, but are not limited to, myocardial infarction, stroke, sickle cell disease, diabetes, β-thalassemia, cancer, antiphospholipid syndrome, endotoxemia, uremia, malaria, sepsis, hepatitis, ischemia-reperfusion, blood storage lesions, Alzheimer's disease, Duchenne muscular dystrophy, eclampsia, glucose-6-phosphate dehydrogenase deficiency, and Wilson's disease. Yet despite the observation of PS exposure in these human diseases, using PS exposure as a therapeutic target has received little attention.

More recently, I used flow cytometry to examine horse erythrocytes for possible PS exposure. Even though work to date has involved blood from horses “at rest” (n=6), not from exercise, it was unexpectedly found that, in all horses, 8-20% of the blood cells stained positive for annexin V^{FITC}, a fluorochrome specific for PS, indicating that PS exposure indeed occurs “naturally” on equine erythrocytes. This result corresponds to an identical result in a pilot study performed in 1996 in which flow cytometry identified PS exposure in one horse. Thus, an increased number of erythrocytes with PS exposure appears to correlate with the increase in echinocytosis in exercising horses and would, therefore, follow Kamp et al. (2001) evidence revealing that echinocytes are cells with PS exposure. This evidence suggests that the number of erythrocytes with PS exposure would be much greater in exercising horses than that of horses at rest (8-20%).

Wood, B. L. et al. (Blood 88:1873-1880, 1996) reported that 0.4-12.0% of erythrocytes of (human) patients (n=205) with sickle cell disease (SCD) stained positive for annexin V^{FITC}. Wood et al. further documented that their results were consistent with other reports of PS exposure in SCD patients. Thrombosis is a risk factor of SCD. Therefore, the greater number of erythrocytes with PS exposure in horses than in human SCD patients suggests that horses are at great risk of thrombosis and microthromboembolic crisis during exercise.

Also, I unexpectedly found annexin V^{FITC} fluorescence of cellular membrane microparticles. Membrane microparticles, microfragments of membrane, are known to contain a high phosphatidylserine content. They are recognized as amplifiers of coagulation and, therefore, harmful. Notably, the PS molecule is not species specific, therefore, its assessment in humans applies also to horses, and vice versa. (Freyssinet, J.-M. J. Thromb. Haemost. 1:1655-1662, 2003). Therefore, echinocytes with exposed PS combined with cell fragmented microparticles containing PS exposure creates a thrombotic hazard to horse health. Flow cytometry evaluation of these submicroscopic (0.2-2.0 μm) membrane particles is just emerging as a new in vivo diagnostic marker (in human medicine) for ongoing subclinical pathophysiology.

The interplay between PS exposure and echinocyte development involves multiple, inter-related pathways, including, but not limited to, ATP-depletion and increased intracellular calcium concentration. Echinocyte development starts with a reduction of cellular energy (ATP) that causes malfunction of enzymatic transporters and other proteins of the red blood cell membrane. ATP-depletion can be induced slowly by incubating whole blood at 37° C. Cascading catabolic events result from the inactivation of the ATP-dependent, membrane calcium pump, resulting in increased intracellular calcium concentration. As discussed above, the increased intracellular calcium leads to the inactivation of translocase and floppase and the activation of scramblase, calpain, and the Gardos channel, causing PS exposure and leading to an abnormal cell shape (e.g., echinocytes).

Oxidative stress may damage enzymatic transporters and other proteins of the red blood cell membrane and initiate the same events as those resulting from ATP-depletion. Oxidative stress may also cause excess energy utilization as a compensating mechanism in an attempt to restore damaged membrane enzymes.

Lysophosphatidic acid (“LPA”) induces thrombogenic activity through phosphatidylserine exposure and procoagulant microvesicle generation in human erythrocytes (Chung, S-M, et al. Arteriosclerosis, Thrombosis, and Vascular Biology 27:414-421, 2007). Although LPA can directly induce the influx of intracellular calcium (33), LPA-induced echinocytes, PS exposure microvesicle generation, increased thrombin generation, and endothelial adherence occurs by a mechanism independent of excess intracellular calcium and G protein-coupled receptor-mediated pathway. Protein kinase C (“PKC”) ζ, activated by LPA, is responsible for PS exposure and related changes in red blood cells (38). PKCζ is independent of intracellular calcium (48). PKC activation can influence cytoskeletal integrity and erythrocyte function by phosphorylation of membrane protein (e.g., band 4.1, 4.9, and adducin (49-51), which could mediate LPA-induced changes in erythrocytes. Chung S-M et al. showed that PKC inhibitors significantly reduced PS exposure and microvesicle generation induced by LPA. LPA, released from activated platelets during coagulation, can diffuse directly into neighboring cells, activating intracellular signaling pathways via an LPA receptor (52). LPA has been shown to inhibit flippase directly, independent of excess intracellular (Chung, S-M, et al., Arteriosclerosis, Thrombosis, and Vascular Biology 27:414-421, 2007). Exposing red blood cells to LPA resulted in PS exposure and increased adherence to endothelial cells and potentiated thrombin generation and accelerated coagulation as a result of echinocyte PS exposure (Chung, S-M, et al. Arteriosclerosis, Thrombosis, and Vascular Biology 27:414-421, 2007). Chung, S-M, et al. were the first to report on the procoagulant microvesicle generation by an endogenous substance contained in normal erythrocytes.

Other naturally-occurring compounds that may be involved in the interplay between PS exposure and echinocyte development, or other hemorheologic abnormalities include, but are not limited to, arachadonic acid (Chung, S-M, et al. Arteriosclerosis, Thrombosis, and Vascular Biology 27:414-421, 2007), prostaglandin E₂ ((Chung, S-M, et al. Arteriosclerosis, Thrombosis, and Vascular Biology 27:414-421, 2007; Kaestner, Prostaglandin E2 activates channel-mediated calcium entry in human erythrocytes: an indication for a blood clot formation supporting process. Thromb Haemost. 92:1269-72, 2004; Lang, P. A. et al. PGE₂ in the regulation of programmed erythrocyte death. Cell Death Differ. 12:415-428, 2005), phospholipase A₂, secretory phospholipase A₂ Neidlinger, N. A. et al., Hydrolysis of phosphatidylserine-exposing red blood cells by secretory phospholipase A₂generates lysophosphatidic acid and results in vascular dysfunction. J Biol Chem 281:775-781, 2006), platelet-activating factor, lipopolysaccharide, ceramide (Gulbins, E. Regulation of death receptor signaling and apoptosis by ceramide. Pharmacol Res. 47:393-9, 2003; Lang, K. S. et al. Involvement of ceramide in hyperosmotic shock-induced death of erythrocytes. Cell Death Differ. 11:231-43, 2004), adrenakine and noradrenaline (Hilario, S., An in vitro study of adrenaline effect on human erythrocyte properties in both gender. Clin Hemorheol Microcirc. 28:89-98, 2003), lysolecithin, bile acid, lactate, picric acid (Sheremet'ev IuA, et al., [A study of the aggregation of human red blood cells induced by picric acid]. [Russian] Biofzika 50:901-902, 2005), glycosylphosphatidylinositol-anchored proteins (Smrz, D. et al. Non-apoptotic Phosphatidylserine Externalization Induced by Engagement of Glycosylphosphatidylinositol-anchored Proteins J Biol. Chem. 282:10487-10497, 2007), and n-ethylmaleimide (Kuypers, F. A. et al., Detection of altered membrane phospholipid asymmetry in subpopulations of human red blood cells using fluorescently labeled annexin V. Blood 878:1179-1187, 1996).

Kuypers showed that N-ethylmaleimide (“NEM”) blocks the activity of aminophospholipid translocase (flippase) by complexing a sulfhydryl group necessary for its activity. Thus NEM prevents the rapid transport of exposed PS back into the inner lipid membrane monolayer. Membrane scrambling, by NEM alone, does not occur unless coupled with excess intracellular calcium. The combination of NEM and excess intracellular calcium leads to the development of echinocytes. Conversely, calcium plus the A23187 ionophore, without NEM, generated PS exposure, but to a lesser degree than when NEM was added (Kuypers, F. A. et al., Blood 878:1179-1187, 1996), showing the beneficial effect of an active translocase in keeping PS exposure in check. Accordingly, the combination of an inactivated aminophospholipid translocase and excess intracellular calcium causes an irreconcilable maldistribution of PS that perturbs the membrane structure. The outer monolayer surface area disproportionately expands relative to the inner monolayer surface area (due to relatively greater number of lipid molecules) and, in accordance with the “bilayer-couple hypothesis”, the cell transforms into an abnormal shape with spicules (echinocyte). The spicules may fragment (pinch-off) and circulate as vesicles with a high concentration PS exposed on the surface. PS exposure also appears to be associated with red blood cell aggregation. High phosphodiesterase (“PDE”) activity works coordinately with intracellular calcium entry. PDE activity may regulate membrane delimited cAMP concentrations that are important for control of cell-cell interaction and red blood cell aggregation. Inhibiting PDE with dibutyryl-cAMP, a cell permeable cAMP analog that preferentially activates cAMP-dependent protein kinases (e.g., protein kinase A), inhibits intracellular calcium entry, prevents PS exposure and reduces red blood cell aggregation by about 50% in vitro (Muravyov, A. V, et al. Hemorheological efficiency of drugs, targeting on intracellular phosphodiesterase activity: in vitro study (Clin Hemorheol Microcirc. 36:327-34, 2007).

3. Adhesion Molecules

Blood cells (RBCs, platelets, leukocytes), endothelial cells, and plasma contain adhesion molecules which, when stimulated, interact with their individual specific receptors and reversibly bind cells-to-cells, cells-to-protein, and protein-to-protein. Adhesion molecules interact very little in health, but upon stimulation by cytokines (primarily TNFa, IL-1, IL-6, and IL-8) in disease, or in non-disease stress, adhesion molecules can create said interactions (“stickiness”) of blood cells that bind in aggregates with an intensity (tightness) or magnitude (size) that can impede or block blood flow in the microcirculation. Adhesion molecules can also generate blood cell-to-endothelial cell adhesion that can impede or block blood flow within microcirculatory vessels. The adhesion molecules, listed herein, according to cell adhesion type include, but are not limited, to:

1. Blood Cells-to-Blood Cells: for example, a fibrinogen protein, an immunoglobulin (e.g., IgG, IgA, IgM), a thrombospondin protein, a PS-β₂GP1-immunoglobulin complex (as occurs in antiphospholipid syndrome), a fibronectin protein, a fibrinogen protein, a von Willebrand protein, a C-reactive protein etc.

2. Blood Cells-to-Endothelial Cells: for example, an I-CAM-1 and I-CAM-2 protein, a V-CAM-1 protein, a PECAM-1 protein, an integrin protein (e.g., VLA-4, LFA-1, Mac-1 antigen), a selectin protein (e.g., P-selectin, L-selectin, or E-selectin), a cadherin protein (e.g., E-cadherin), a cluster of differentiation (CD35, CD36), an addressin protein (MAdCAM-1), etc.

### Therapeutic Applications

During intense exercise, such as racing, vasodilation is maximal and does not change, i.e., blood vessels can compensate no further, so hemorheology becomes the only regulator of blood flow to tissues. Therefore, increased blood viscosity generated by an increase in any hemorheologic determinant or a thrombotic state would increase blood flow resistance, resulting in increased generalized or localized ischemia, degeneration, and/or necrosis of tissue supplied by the systemic circulation, in the systemic system, or increased pulmonary hypertension in the pulmonary system. These pathophysiological phenomena, if left untreated, can result in disease.

Thrombosis (blood clots) or microthromboemboli (tiny clumps of blood components; micro clots) or an increase in any hemorheologic determinant in a mammal can impede or inhibit flow in microcirculatory blood vessels and lead to systemic or pulmonary diseases or to premature fatigue or reduced performance due to hemorheopathy (abnormal mechanics of blood flow). Through either the pulmonary or systemic circulation, blood flows to all cells of the body and nourishes every organ in the body. The pulmonary circulation carries deoxygenated blood away from the heart, to the lungs, where blood is oxygenated and delivered back to the heart. The systemic circulation carries the oxygenated blood away from the heart and delivers it to all the cells of the body through the arterial system. It also carries deoxygenated blood through the venous system back to the heart. Therefore, no organ system is exempt from damage or disease caused by the effects of hemorheopathy.

A hemorheologic abnormality may be associated with an infectious disease, including but not limited to a bacterial infection, including, but not limited to Clostridium perfringens, Brucella abortus, Anaplasm phagocytophilium, Pasteurella haemolytica, Haemophilus parasuis, Streptococcus spp., Vibrio parahemolyticus, Listeria monocytogenes, Leptospira interrogans serovar Pomona, Helicobacter pylori, etc.; a viral infection, including, but not limited to, cytomegalovirus, herpes simplex virus, ebola virus, etc.; or a parasite infection, including, but not limited to a sporazoan parasite (e.g., Plasmodium (malaria), Babesia spp., etc.), a protozoan parasite (e.g., Trypanosoma (T. Congolese, T. vivax, T cruzi), Leishmania spp., Theileria Serengeti, etc.), or a metazoan parasite (e.g., Hydra vulgaris). A hemorheologic abnormality can also be associated with the venom of an insect (e.g., scorpion), a spider, a cnidarian, or a snake (e.g., rattlesnake, cobra, coral snake), or a toxin (endotoxin (e.g., endotoxins produced by Haemophilus parasuis, Salmonella typhi, Escherichia coli, etc.), red maple (Accer rubrum), etc.).

Thus, certain embodiments provide a method of treating a hemorheologic abnormality that is associated with a pulmonary disease, including, but not limited to pulmonary hypertension (arterial and venous), intrapulmonary right-left shunting, hypoxemia, exercise-induced pulmonary hemorrhage, fibrosis, hemosiderosis, vascular remodeling (hyperplasia) or partial or complete veno-occlusion (arterial or venous).

Other embodiments provide a method of treating a hemorheologic abnormality that is associated with a systemic disease, including, but not limited to, myopathy (muscle disease), laminitis, navicular disease, gastric ulcers, subepiglottic ulcers, bone demineralization, musculoskeletal failure, bone cysts (aseptic necrosis), laryngeal hemiplegia, cardiac arrhythmias, myocardial fibrosis, degenerative joint disease, degenerative myeleoncephalopathy, osteochondrosis, liver disease, kidney disease, vascular wall damage, vascular remodeling (hyperplasia). In certain embodiments, the systemic disease is caused by an infectious disease, including but not limited to a bacterial infection, a viral infection or a parasite infection of a venom or a toxin. In certain embodiments, the systemic disease is associated with the formation of echinocytes, and includes, for example, myocardial infarction, stroke, sickle cell disease, diabetes, β-thalassemia, cancer, antiphospholipid syndrome, endotoxemia, uremia, malaria, sepsis, hepatitis, ischemia-reperfusion, blood storage lesions, Alzheimer's disease, Duchenne muscular dystrophy, eclampsia, glucose-6-phosphate dehydrogenase deficiency, or Wilson's disease.

The methods of treating a hemorheologic abnormality associated with a pulmonary or systemic disease comprise administering to a subject an effective amount of a hemorheologically-active compound. In certain embodiments, the subject is a non-human mammal, including, but not limited to, a horse, a cow, a sheep, a pig, a dog or a cat. In other embodiments, the subject is a human. In other embodiments, the method further comprises (a) obtaining data about the one or more hemorheologic determinants in the blood of said subject before and after administering said hemorheologically-active compound to said subject; and (b) analyzing the data about the one or more hemorheologic determinants to assess the effectiveness of administering said hemorheologically-active compound to said subject.

An increase in a hemorheologic determinant in a mammal can impede or inhibit flow in microcirculatory blood vessels, which supply blood to the body's organs, including, but not limited to, the muscles, resulting in tissue oxygen deprivation and premature fatigue and reduced performance capacity, particularly when the mammal is exposed to a stress. Thus, certain methods are directed to treating a hemorheologic abnormality in a mammal, such as a human, wherein the hemorheologic abnormality occurs in the blood of said mammal after experiencing a stress sufficient to induce the hemorheologic abnormality, the blood of said human having a normal level of a hemorheologic determinant prior to experiencing said stress, the method comprising administering to said mammal an effective amount of a hemorheologically-active compound. In certain embodiments, the stress is a non-disease related stress, including, but not limited to, exercise, exposure to a hot and humid environment, skin burn, exposure to high altitude, underwater diving, hypoxia, surgery, blood storage lesions, or space travel. In other embodiments, the hemorheologic abnormality is an increased blood viscosity and the blood of said mammal has a normal viscosity prior to experiencing the non-disease related stress.

Exercise

As compared to other species, healthy horses uniquely generate a far greater blood hyperviscosity due to stress, such as exercise. In exercising horses, all hemorheologic determinants increase significantly. One of the determinants, abnormal red blood shape (e.g., echinocytes), also leads to echinocyte adherence to endothelial cells and membrane fragmentation (microparticle formation), a procoagulant condition generating microthromboemboli, both of which adversely affect blood flow. Abnormally increased blood viscosity at levels much less than those generated by exercising horses, are known to cause a resistance to microcirculatory blood flow that induce ischemia, degeneration, or necrosis of tissue supplied by the systemic circulation, or pulmonary hypertension in the pulmonary circulation resulting in pulmonary or systemic disease or reduced oxygenation to tissues and decreased performance capacity. Accordingly, animals, such as horses, are at risk of acquiring a disease “silently” (without recognizable signs or symptoms) merely by intense exercise.

In one embodiment, the hemorheologic abnormalities in exercising animals, such as horses, result in pulmonary circulatory dysfunction, leading to hypoxemia and pulmonary hemorrhage. The increased severity in hemorheologic determinants and the ensuing pulmonary pathophysiology during exercise suggest that multiple pathologic blood rheology mechanisms operate synergistically to induce blood hyperviscosity. Blood hyperviscosity, in turn, impedes or blocks microcirculatory blood flow and, thereby, increases flow resistance, thus leading to the development of pulmonary hypertension and a three-pronged pathway to hypoxemia. (Boucher, J. H. and P. Connes. HORSES: Ideal hemorheological models of HUMAN exercise pathophysiology. Oral presentation at the International Conference on Clinical Hemorheology, 2008).

The first phenomenon leading to hypoxemia is capillary fluid leakage. Capillary fluid leakage leads to interstitial edema, causing an oxygen diffusion limitation from the alveoli to blood in capillaries. Second, the pulmonary hypertension triggers a pressure-release mechanism that induces pulmonary R-L shunting to protect against capillary rupture, thereby, contributing to hypoxemia by diluting the oxygenated blood with deoxygenated blood. Finally, if the intrapulmonnary shunting (pressure-release mechanism) is not sufficient to compensate for the hypertension, capillary membrane disruption would cause blood to accumulate in the interstitial spaces and alveoli. The extravascular blood exacerbates hypoxemia by further impinging upon oxygen diffusion. Also, extravascular blood from ruptured capillaries leads to the well-known disease of horses, exercise-induced pulmonary hemorrhage (EIPH). This disease has recently been recognized in human athletes also.

Indeed, exercise in healthy humans also generates increases in hemorheologic determinants, but the hemorheologic abnormalities occur to a far lesser extent than those in horses, as indicated in the table below.

| | | % Increase from | |
|---|---|---|---|
| Determinant of | | Rest After Exercise | |
| Blood Viscosity | Reference cited below | Horse | Human |
| RBC Concentration | 5, 6, 9, 13, 18, 21, 25, 26, | 62 | 10 |
| | 27, 31, 32, 33, 36 | | |
| RBC Aggregation | 8, 9, 12, 13, 18, 21, 22, 23, | 205 | 8 |
| | 25, 27, 32, 33, 34, 36 | | |
| RBC Deformability | 5, 6, 9, 12, 13, 18, 21, 22, | 100 | 15 |
| | 23, 25, 27, 29, 31, 33, 34, 35 | | |
| Plasma Viscosity | 8, 9, 22, 23, 25, 26, 27, 32, | 9 | 10 |
| | 33, 34, 36 | | |
| RBC Shape Change | 5, 6, 18, 30 | 60 | 6 |
| to Echinocytes | | | |

The following references report on one or more abnormal blood viscosity determinant induced by exercise in horses or humans:

HORSES

| | |
|---|---|
| 1 | ALLEN, BV. Vet Rec. 122: 329-32, 1988. |
| 2 | AMIN, T. M. and J. A. Sirs. The blood rheology of man and various |
| | animal species. Qtly. J. Exp. Physiol. 70: 37-49, 1985. |
| 3 | BASKURT, O. K. R. A. Farley & H. J. Meiselman. Erythrocyte |
| | aggregation tendency and cellular properties in horse, human, and rat: |
| | A comparative study. Am J Physiol. 273(6 Pt 2): H2604-2612, 1997. |
| 4 | BASKURT, O. K. & H. J. Meiselman. Susceptibility of equine |
| | erythrocytes to oxidant-induced rheologic alterations. Am J Vet Res. 60: |
| | 1301-1306, 1999. |
| 5 | BOUCHER, J. H., E. W. Ferguson, C. L. Wilhelmsen, N. Statham, and |
| | R. R. McMeekin. Erythrocyte alterations during endurance exercise in |
| | horses. J. Appl. Physiol. 51: 131-134, 1981. |
| 6 | BOUCHER, J. H. Exercise-induced echinocytosis. Jones, W. E. (ed.), |
| | Equine Sports Medicine, Chapter 4. Philadelphia: Lea & Febiger; 1988. |
| 7 | BRAASCH, D. Flow properties in the microcirculation. Hemodilution: |
| | Theoretical basis and clinical application. Int. Symp. Rottach-Egemn, |
| | Karger, Basel, 1972, pp. 57-58. |
| 8 | COYNE, C. P., G. P Carlson, M. S. Spensley, and J. Smith. Preliminary |
| | investigation of alterations in blood viscosity, cellular composition, and |
| | electrophoresis plasma protein fraction profile after competitive racing |
| | activity in Thoroughbred horses. Am. J. Vet. Res. 51: 1956-1963, 1990. |
| 9 | CATALANI, G. et al. Acute training in racing horses at two different |
| | levels of effort: A hemorheological analysis. Clin Hemorheol & |
| | Microcirc 37: 245-252, 2007. |
| 10 | DINTENFASS, L. & Liao F-1. Plasma and blood viscosities, and |
| | aggregation of red cells in racehorses. Clin. Phys. Physiol. Meas. 3: |
| | 293-301, 1982. |
| 11 | FAHRAEUS, R. The suspension stability of the blood. Physiol Rev |
| | 9: 241-274, 1929. |
| 12 | FEDDE, M. R. and H. H. Erickson. Increase in blood viscosity in the |
| | sprinting horse: Can it account for the high pulmonary arterial pressure? |
| | Equine Vet. J. 30: 329-334, 1998. |
| 13 | GEOR, R. J., D. J. Weiss, and C. M. Smith II. Hemorheologic |
| | alterations in Thoroughbred horses induced by incremental treadmill |
| | exercise. Am J Vet Res 55: 854-861, 1994. |
| 14 | JOHNN, H. et al. (Rampling). A comparison of the viscometric |
| | properties of the blood from a wide range of mammals. Clinical |
| | Hemorheology 12: 639-647, 1992. |
| 15 | KAIBARA, M., Date, M. and E. Fukada. Dynamic evaluation of |
| | aggregation and agglutination of red blood cells. Biorheology Suppl. |
| | 1: 43-47, 1984. |
| 16 | KUMARAVEL, M. and M. Singh. Sequential analysis of erythrocyte |
| | aggregation in health and diseases. Clin Hemorheol Microcirc. |
| | 17(4): 319-24, 1997. |
| 17 | LIAO, F-1 and L. Dintenfass. Effect of microrheology of blood on the |
| | apparent flow instability in a rotational viscometer. Biorheology |
| | 20: 327-342, 1983. |
| 18 | MCCLAY, C. B., D. J. Weiss, C. M. Smith II, & B. Gordon. |
| | Hemorheologic parameters in the racing Thoroughbred: Implication |
| | for exercise-induced pulmonary hemorrhage. Am. J. Vet. Res. 53: |
| | 1380-1385, 1992. |
| 19 | PERSSON S. Acta Vet Scand. 1967: Suppl 19: 9-189. |
| 20 | POPEL A S, Johnson P C, Kameneva M V, Wild M A. C J Appl |
| | Physiol. 77: 1790-4, 1994. |
| 21 | SOMMARDAHL C S, Andrews F M, Saxton A M, Geiser D R, |
| | Maykuth P L. Am J Vet Res. 55: 389-394, 1994. |
| 22 | STOIBER, B., C. Zach, B. Izay and U. Windberger. Whole blood, |
| | plasma viscosity, and erythrocyte aggregation as a determining factor |
| | of competitiveness in standard bred trotters. Clin Hemorheol |
| | Microcirc. 32: 31-41, 2005. |

HUMANS

| | |
|---|---|
| 23 | AJMANI, R. S. et al. Oxidative stress and hemorheological changes |
| | induced by acute treadmill exercise. Clin Hemorheol Microcirc 28: |
| | 29-40, 2003. |
| 24 | BASKURT, O. K., R. A. Farley and H. J. Meiselrnan, Erythrocyte |
| | aggregation tendency and cellular properties in horse, human, and rat: |
| | A comparative study, Air. J. Physiol. 273(6 Pt 2) (1997), H2604-H2612. |
| 25 | BOUCHER, J. H. and P. Connes. HORSES: Ideal hemorheological |
| | models of human exercise pathophysiology. (Publication in Progress, |
| | 2009). |
| 26 | BRUN, J. F. C. Fons, C. Supparo, C. Mallard and A. Orsetti, Could |
| | exercise-induced increase in blood viscosity at high shear rate be |
| | entirely explained by hematocrit and plasma viscosity changes?, |
| | Clin. Hemorheol. 13 (1993), 187-199. |
| 27 | BRUN, J.-F., et al. The triphasic effects of exercise on blood rheology: |
| | which relevance to physiology and pathophysiology? Clin Hemorheol |
| | Microcirc. 19: 89-104. 1998. |
| 28 | BRUN, J.-.F., E. Varlet-Marie and I. Aloulou. Hemorheologic alterations |
| | of training and overtraining. (In Press, Biorheology, 2010). |
| 29 | CONNES, P., D. Bouix, F. Durand, et al. Is hemoglobin desaturation |
| | related to blood viscosity in athletes during exercise. Int. J. Sports Med. |
| | 25: 569-574, 2004. |
| 30 | CONNES, P. and J. H. Boucher. Echinocytosis in athletes with |
| | exercise-induced hypoxemia, Clinical Hemorheology and |
| | Microcirculation, 44: 107-114, 2010. |
| 31 | ERNST, E., L. Daburger and T. Saradeth. The kinetics of blood |
| | rheology during and after prolonged standardized exercise. Clinical |
| | Hemorheology 11: 429-439, 1991. |
| 32 | GUEGUEN-DUCHESNE, M., F. Durand, J. Beillot, et al. Effect of |
| | maximal physical exercise on hemorheological parameters in top level |
| | sportsmen. Clinical Hemorheology 9: 625-632, 1989. |
| 33 | MONCHANIN, G., P. Connes, D. Wouassi, A. Francina, B. Djoda, P. E. |
| | Banga, F. X. Owona, P. Thiriet, R. Massarelli and C. Martin, |
| | Hemorheology, sickle cell trait, and alpha-thalassemia in athletes: |
| | effects of exercise, Med. Sci. Sports E: rerc. 37 (2005). |
| 34 | NAGESWARI, K. et al. Effects of exercise on rheological and |
| | microcirculatory parameters. Clin Hemorheol Microcirc. 23: 243-7, 2000. |
| 35 | TRIPETTE, J., M. D. Hardy-Dessources, F. Sara, M. Montout- |
| | Hedreville, C. Saint-Martin, O. Hue and P. Connes, Does prolonged |
| | and heavy exercise impair blood rheology in sickle cell trait carriers?, |
| | Clin. J. Sports Med. 17 (2007), 465-470. |
| 36 | VARLET-MARIE, E. et al. Reduction of red blood cell disaggregability |
| | during submaximal exercise: relationship with fibrinogen levels. Clin |
| | Hemorheol Microcirc. 28: 139-49, 2003. |

Two reports cited that human athletes had a small but significantly increased number of echinocytes during strenuous exercise (Selby, G. B. et al., Athlete's echinocytes: New cause of exertional hemolysis. Blood 70:56a, 1987; Connes, P. and J. H. Boucher, Echinocytosis in athletes with exercise-induced hypoxemia Clinical Hemorheology and Microcirculation, 44:107-114, 2010). Curiously, the low order of increased echinocytes (7-10% of the total cells) in exercising humans presumably dissuades serious attention to echinocytosis in human medicine and draws skepticism about the clinical significance of such cells.

Although a 6% increase in echinocytes observed in human athletes after intense exercise may seem insignificant, it has been shown that an echinocyte population as low as 2.5 percent of the total population caused a significantly reduced filtration time through capillary-sized pores (Pasquini, G. et al., A small sub-population of stiff red cells modifies the erythrocyte filtration test, Clin. Hemorheol. 4:495-503, 1985). Likewise, Dintenfass, Clin. Hemorheol. 6, 435-437, 1986, noted that the small increase of echinocytes (6-7% of the total RBC population) in astronauts at the end of Apollo missions (early 1970s), was significant. He speculated that the echinocyte rigidity impeded microcirculatory blood flow in bone, caused local areas of bone absorption, and led to the osteoporosis observed in the astronauts. Yet despite these observations, Dintenfass did not propose any treatment for the slight echinocytosis observed in astronauts. Nor did NASA publish further on the topic. The small population of echinocytes generated by human athletes may impede blood flow and reduce tissue oxygenation in much the same way as do small populations of rigid sickle cells (Chien, S. Rheology of sickle cells and the microcirculation, N. Engl. J. Med. 311, 1567-1569, 1984). For example, by remaining at the precapillary site for longer periods than cells with normal deformability, they would disproportionately occupy most of the capillaries and reduce flow to a much greater extent than would be expected on the basis of their percent of the total red cell population. Thus, preventing even low concentrations of echinocytes should improve microcirculatory blood flow and enhance disease recovery.

In one embodiment, the method is directed to treating increased blood viscosity in a mammal that occurs after exercise, and the method comprises administering to said mammal an effective amount of a hemorheologically-active compound, wherein administering said hemorheologically-active compound reduces an increase in blood viscosity that occurs in the blood of said mammal after exercise sufficient to induce increased blood viscosity, the blood of said mammal having a normal viscosity prior to said exercise.

In another embodiment, the method is directed to treating hypoxemia or pulmonary hypertension that occurs after exercise, and the method comprises administering to a mammal an effective amount of a hemorheologically-active compound, wherein administering said hemorheologically-active compound treats hypoxemia or pulmonary hypertension that occurs in the blood of said mammal after exercise sufficient to induce hypoxemia or pulmonary hypertension.

### Mammals

Mammal includes without limitation any members of the Mammalia. A mammal, as a subject or patient in the present disclosure, can be from the family of Primates, Equidae, Camivora, Proboscidea, Perissodactyla, Artiodactyla, Rodentia, and Lagomorpha. Among other specific embodiments a mammal of the present invention can be Canis familiaris (dog), Fells catus (cat), Elephas maximus (elephant), Equus caballus (horse), Sus domesticus (pig), Camelus dromedarious (camel), Cereus axis (deer), Giraffa camelopardalis (giraffe), Bos taurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), or Homo sapiens (human). In a particular embodiment, the mammal is a human. In other embodiments, the mammal is a non-human mammal animal, including mammals raised on farms for consumption by humans (e.g., cows, sheep, goats, pigs) or animals of social importance to humans, such as animals kept as pets (e.g., horses, dogs, cats, etc.) or in zoos.

While hemorheologic abnormalities in horses and humans have drawn the most attention to date, evidence suggests that other mammals may experience hemorheologic abnormalities in their blood, either in association with a pulmonary or systemic disease or in response to a non-disease related stress, including, but not limited to, exposure to a hot and humid environment, skin burn, exposure to high altitude, underwater diving, hypoxia, surgery, or space travel.

For example, the physiological stress of calving and subsequent lactation may cause a syndrome in cows characterized by ATP depletion of red cells and intravascular hemolysis (Ogawa, E. et al., Bovine postparturient hemoglobinemia: Hypophosphatemia and metabolic disorder in red blood cells. Am. J. Vet. Res. 48:1300-1303, 1987). Metabolic (ATP) depletion of the red cells classically induces echinocytes, a fragile cell abnormality capable of hemolysis. A diagnosis of hypophosphatemia, based upon low serum phosphorus levels, warranted treatment with inorganic phosphate for 2-5 days. Treating with inorganic phosphate resulted in an absence of hemolysis, and complete recovery within 14-21 days (Ogawa, E. et al., Am. J. Vet. Res. 48:1300-1303, 1987).

Hemolytic anemias, originating from an infectious, parasitic, or toxic origin, or due to antigen-antibody reactions, can cause red cell membrane molecular alterations and result in crenation, with echinocytes numbering up to 90 percent of the observed red cells (Doxey, D. L. Hemolytic anemias. In: Clinical pathology and diagnosticprocedures; Bailliere Tindall, London, Second Edition, 1983, pp. 179-182; Thompson, J. C. Morphological changes in red cells of calves caused by Leptospira interrogans serovar pomona. J. Comp. Path, 96:517-527, 1986). That degree of severity could cause an ischemic “crisis” to organ systems similar to the sickle cell “crisis” that occurs in humans suffering from sickle cell disease and could lead to death, in part, from ischemic cellular damage to affected organs systems. There are no literature reports that suggest any treatment, palliative or otherwise, to combat the echinocytic sequela. Treating such a condition with a hemorheologically-active compound, such as an ATP enhancing drug, could alleviate the echinocytosis, providing a major therapy breakthrough in diseases that have no current mode of treatment.

High numbers of echinocytes have also been found in the blood of greyhounds after strenuous exercise (Bjotvedt, G. et al., Strenuous exercise may cause health hazards for racing Greyhounds. Veterinary Medicine (December): 1481-1487, 1984) and in dogs of all breeds with specific diseases (uremia, a variety of malignancies, immune-mediated anemia, etc.) (Weiss, D. J. et al., Quantitative evaluation of echinocytes in the dog. Veterinary Clinical Pathology 19:114-118, 1991).

### Hemorheologically-Active Compounds

A hemorheologically active compound is one that improves blood flow by reducing an abnormal level of a hemorheological determinant. Based on an elucidation of the multiple, interrelated hemorheologic pathways, a number of different compounds have been identified that help to maintain blood homeostasis by:

1) optimizing blood viscosity through:
a) improving red blood cell deformability;
b) decreasing abnormal red blood cell aggregation;
c) inhibiting polycythemia or anemia;
d) decreasing elevated plasma viscosity; or
e) maintaining normal red blood cell shape (e.g., preventing the formation of echinocytes, stomatocytes, or elliptocytes);

2) modulating, inhibiting, or preventing increased resistance to blood flow;

3) contributing to drag reduction of blood structures (drag reducing agents);

4) contributing to antithrombotic activity;

5) contributing to thrombolytic activity;

6) contributing to antithrombogenic activity (inhibiting or preventing hypercoagulation states);

7) contributing to coagulation balance (the balance between coagulation and excessive anticoagulation);

8) regulating vascular adhesion molecules (inhibiting or preventing blood cell adhesion to the blood vessel endothelium);

9) normalizing membrane and intracellular biochemistry of blood cells; or

10) modulating or inhibiting or preventing blood cell activation.

Hemorheologically active compounds can be classified into five main groups: a plasma membrane stabilizing compound, an anticoagulant compound, a drag reducing compound, an adhesion molecule inhibitor, and a cytokine inhibitor.

Plasma membrane stabilizing compounds are compounds that help to stabilize the structure of the plasma membrane of a cell, including, but not limited to, a compound that increases adenosine deaminase activity (e.g., PEG-conjugated adenosine deaminase); a compound that increases intracellular ATP (e.g., vinpocetine, drotaverine, pentoxifylline, denbufylline, torbafylline, 3-isobutyl-1-methylxanthine, dibutyryl cAMP, cyclandelate, cilostazol, AMP-activated protein kinase, 5-aminoimidazole-4-carboxamide-1-β-D-ribofuranoside (AICAR), metformin, GW1516, fructose diphosphate, ATP-MgCl₂, S-adenosyl-L-methionine (SAM), or naftidrofuryl); an antioxidant (e.g., α, β, γ, or δ tocopherol, α, β, γ, or δ tocotrienol, genistein, CBLB613, etoposide, thioredoxin, furosemide, resveratrol, xanthohumol, zidovudine, thymol, BIO 300, trolox, ascovertin, dithiothreitol, IRFI 042, N-acetylcysteine, pilloridine dithiocarbamate, pirfenidone, dipyridamole, picroliv, L-carnitine, reduced glutathione (GSH), monoHER (7-monohydroxyethylrutoside), phenylbutyrate, diphenyleneiodonium, pyruvate, EUK189, a fullerene, ceruloplasmin, transferrin, uric acid, superoxide dismutase, catalase, or glutathione peroxidase); an ion channel inhibitor (e.g., piracetam, bepridil, nifedipine, nitrendipine, clotrimazole, ICA-17043, amiloride, ethylisopropylamiloride, naftidrofuryl, cepharanthine, verapamil, diltazem, NS3623, magnesium pidolate, magnesium chloride, ω agatoxin TK, or a calcium ATPase inhibitor); a compound that inhibits PS exposure (e.g., hydroxyurea, L-arginine, haptoglobin, N-a-tosyl-L-lysine chloromethyl ketone (TLCK), prothrombin RR157/R268A, annexin V, diannexin, an antibody directed against thrombospondin (TSP), high density lipoprotein (HDL), apolipoprotein A1, ethanimidothioc acid (RS421), dithioerythritol, nafamostat mesilate (FUT-175), CGS12970, CGS13080, glutamine, s-adenosyl-L-methionine (SAM), an antibody directed against phosphatidylserine, tetrathiomolybdate, diltiazem, quercetin, catechin, atorvastatin, zidovudine, glyburide); a compound that inhibits apoptosis/eryptosis (eryptosis is the programmed cell death of red blood cells) (e.g., z-DEVD-fmk, HAX-1, cinalukast, ON01210, nicorandil, doxorubicin, serofendic acid, diazoxide, dopamine, tauroursodeoxycholic acid, L-carnitine, propionyl-L-carnitine, dobesilate calcium, cyclodextrin, medroxyprogesterone, thalidomide, revlimid, naftidrofuryl, doxazosin, EDTA); a compound that inhibits calpain (e.g., E64); a compound that inhibits protein kinase C (e.g., calphostin C or chelerythrine), a compound that inhibits phospholipase A₂ (e.g., manoalide, dysidotronic acid, or cyclolinteinone); a compound that inhibits platelet activating factor (e.g., ABT-491 or BB-882); a prostaglandin agonist (e.g., ONO AE1-329); a natural hemorheologic enhancer (e.g., zinc, L-alanine, phosphate, histidine, or taurine); or a phytochemical (e.g., myakuryu, HemoHIM, Lychnis chalcedonica extract, diosmin, hesperidin, danshensu, yunnan baiyao, Pfaffia paniculata extract, oligomeric proanthocyanidins, curcumin, Gingko biloba extract, khelline, paeonia extract, ligustrazine, eburnamonine, octacosanol, pentosan polysulfate, olive leaf extract, piyavit, raubasine, troxerutin, or angelica extract). In one embodiment, the hemorheologically-active compound is compound that helps to stabilize the structure of the plasma membrane of a cell, with the proviso that the compound is not a compound that increases intracellular ATP or an ion channel inhibitor.

Anticoagulant compounds include, but are not limited to, an anti-phospholipid syndrome therapy (e.g., cyprofloxacin); a compound that inhibits microparticle release (e.g., abciximab (a GP IIb/IIIa receptor antagonist), RPR 10989, RPR 110885, cilostazol, cytocalasin D, calpeptin, or protein kinase B); tissue factor or Factor VIIa inhibitors (e.g., hTFAA (a humanized mutant tissue factor made by replacing Lys 165 sand Lys 166 with alanine), G17905, dilazep, dimethyl sulfoxide, or tissue factor pathway inhibitor (TFPI)); a Factor IXa inhibitor (e.g., 10C12, BC2, fondaparinuxm, idraparinux, idrabiotaparinux, otamixaban, DX-9065A, apixaban, rivaroxaban, betrixaban, edoxaban, or TAK-442); a prothrombinase inhibitor (e.g., agkistrodon acutus snake venom, trimeresurus flavoviridis venom, deinagkistrodon acutus snake venom, β2-glycoprotein I, or phospholipase A2 (CM-IV)); a thrombin inhibitor (e.g., hirudin, desirudin, lepirudin, hirulog (bivalirudin), argatroban, ximelagatran, melagatran, BIBR-1049, dabigatran etexilate, AZD0837, or MCC 977); a fibrinogen inhibitor (e.g., tirofibran), a fibrinolytic agent (e.g., nattokinase or defibrotide); activated protein C); or a compound that inhibits P-selectin (e.g., recombinant soluble P-selectin glycoprotein ligand Ig (rPSGL-Ig) or an antibody that binds to P-selection).

Drag reducing compounds include, but are not limited to, polyethylene oxide, aloe vera, hyaluronic acid, or rheothRx.

Adhesion molecule inhibitors include, but are not limited to, an RGD peptide, an antibody that binds to human platelet thrombospondin, CGP69669A, salvianolic acid B, band 3 peptide, anionic polysaccharides, sulfasalazine, or an antibody that binds to an adhesion molecule, including but not limited to, a selectin protein (e.g., P-selectin, L-selectin, or E-selectin), a fibrinogen protein, an immunoglobulin (e.g., IgG, IgA, IgM), an I-CAM protein, a V-CAM protein, an integrin protein (e.g., VLA-4, LFA-1, macrophage-1 antigen), an addressin protein, and a cadherin protein (e.g., E-cadherin). Examples of antibodies that bind to adhesion molecules, include, but are not limited to, anti-Mo-1 (binds to CD11b/CD18), EL-246 (binds to E-selection and L-selectin), anti-avβ₃ (binds to TSP receptor), 7E3 monoclonal antibody (binds to avβ₃ and aIIbβ₃), LM609 monoclonal antibody (binds to avβ₃), and 10E5 monoclonal antibody (binds to allbβ₃).

Cytokine inhibitors include, but are not limited to, inhibitors of TNFa, IL-1, IL-6, and IL-8, as well as, alendonate, EO6 antibody (binds to oxidized phospholipids), 61D3 antibody (binds to monocytes), clodronate, and gadolinium chloride.

### Pharmaceutical Compositions and Methods of Administration

This disclosure provides compositions that are suitable for pharmaceutical use and administration to mammals. The pharmaceutical compositions comprise a hemorheologically-active compound, as described herein, and a pharmaceutically acceptable excipient.

A pharmaceutical composition is formulated to be compatible with its intended route of administration. Methods to accomplish the administration are known to those of ordinary skill in the art. Examples of administration of a pharmaceutical composition include oral ingestion, inhalation, intravenous, intraperitoneal, intramuscular, intracavity, subcutaneous, cutaneous, or transdermal.

Solutions or suspensions used for cutaneous or subcutaneous application typically include at least one of the following components: a sterile diluent such as water, saline solution, fixed oils, polyethylene glycol, glycerine, propylene glycol, or other synthetic solvents; antibacterial agents, such as benzyl alcohol or methyl parabens; antioxidants, such as ascorbic acid or sodium bisulfite; chelating agents, such as ethylenediaminetetraacetic acid (EDTA); buffers, such as acetate, citrate, or phosphate; and tonicity agents, such as sodium chloride or dextrose. The pH can be adjusted with acids or bases. Such preparations may be enclosed in ampoules, disposable syringes, or multiple dose vials.

Solutions or suspensions used for intravenous administration include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Microorganism growth can be prevented using antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin.

Oral compositions include an inert diluent or edible carrier. The composition can be enclosed in gelatin or compressed into tablets. For the purpose of oral administration, the hemorheologically-active compounds can be incorporated with excipients and placed in tablets, troches, or capsules. Pharmaceutically compatible binding agents or adjuvant materials can be included in the composition. The tablets, troches, and capsules, may contain (1) a binder such as microcrystalline cellulose, gum tragacanth or gelatin; (2) an excipient such as starch or lactose, (3) a disintegrating agent such as alginic acid, Primogel, or corn starch; (4) a lubricant such as magnesium stearate; (5) a glidant such as colloidal silicon dioxide; or (6) a sweetening agent or a flavoring agent.

The composition may also be administered by a transmucosal or transdermal route. Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the hemorheologically-active compounds can be delivered in an aerosol spray from a pressured container or dispenser, which contains a propellant (e.g., liquid or gas) or a nebulizer.

The hemorheologically-active compounds arc administered in therapeutically-effective amounts as described. Therapeutically effective amounts may vary with the subject's age, condition, sex, and severity of medical condition. Appropriate dosage may be determined by a physician based on clinical indications. The hemorheologically-active compound-containing composition may be given as a bolus dose to maximize the circulating levels of the hemorheologically-active compounds for the greatest length of time. Continuous infusion may also be used after the bolus dose.

Examples of dosage ranges that can be administered to a subject can be chosen from: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg/kg to 1 mg/kg, 250 μg/kg to 2 mg/kg, 250 μg/kg to 1 mg/kg, 500 μg/kg to 2 mg/kg, 500 μg/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 10 mg/kg to 25 mg/kg, 15 mg/kg to 25 mg/kg, 20 mg/kg to 25 mg/kg, and 20 mg/kg to 30 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration

In certain circumstances, it may be advantageous to formulate compositions in dosage unit form for ease of administration and uniformity of dosage. Dosage unit form as used herein refers to physically discrete units suited for the patient. Each dosage unit contains a predetermined quantity of the hemorheologically-active compound calculated to produce a therapeutic effect in association with the carrier. The dosage unit depends on the characteristics of the hemorheologically-active compounds and the particular therapeutic effect to be achieved.

Toxicity and therapeutic efficacy of the composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀.

The data obtained from cell culture assays and animal studies can be used to formulate a dosage range in humans. The dosage may vary within this range depending upon the composition used and the route of administration. For any hemorheologically-active compound used in the methods described herein, the therapeutically effective dose can be estimated initially using cell culture assays. Animal models can be used to determine a circulating plasma concentrations and IC₅₀ values (i.e., the concentration of hemorheologically-active compounds that achieves a half-maximal inhibition of symptoms). The effects of any particular dosage can be monitored by a suitable bioassay.

### Diagnostic Methods

Until recently, physiologists were unaware of the importance of blood viscosity. With current state-of-the-art equipment and techniques, blood viscosity and other hemorheologic determinants can be measured. Techniques include, but are not limited to, standard microscopy (e.g., brightfield, fluorescent, differential interference contrast), or diffraction phase microscopy, or confocal microscopy, or telemicroscopy of a red blood cell population, including, for example, a blood smear or a wet preparation. Microscopy and telemicroscopy can be used, for example, to analyze the shape of red blood cells and quantify the number of echinocytes, stomatocytes, or elliptocytes. Quantitative red blood cell morphometric analysis software can be used with standard microscopy or telemicroscopy or diffraction phase microscopy, or confocal microscopy to evaluate the percentage of abnormally shaped in a blood sample or the red blood cell geometric and physical characteristic changes (e.g., surface area, volume, circularity) before and after an intervention. These techniques are also useful for evaluating the efficacy of a therapy. Other techniques include blood viscometry for evaluating the absolute and relative viscosity of blood and determining red blood cell aggregation, red blood cell deformability, and plasma viscosity over the physiological shear rate range. Other methods can be used to measure red blood cell aggregation and red blood cell deformability, including, for example, a Lorca or Rheometer. Phosphatidylserine (“PS”) exposure can be measured using techniques, such as, ELISA or flow cytometry, to determine the presence of PS on the cell surface, to measure for a variety of antibody associated antigens (e.g., β₂-GP1, prothrombin) or other membrane proteins (e.g., Band 3 protein) associated with PS exposure, or to measure upstream molecules that lead to PS exposure, including, for example, inflammatory cytokines, such as TNFα, IL-1, or IL-6, or caspase 3 or caspase 8.

Thus, one aspect of the invention is directed to a method of evaluating blood flow in a blood sample from a mammal, the method comprising analyzing said blood sample to determine whether said blood sample comprises a hemorheologic abnormality, wherein the presence of said hemorheologic abnormality in said blood sample indicates reduced blood flow mechanics. In certain embodiments, the method further comprises a step of correlating an indication of reduced blood flow with an increased risk of a pulmonary disease or a systemic disease. In other embodiments, if said hemorheologic abnormality is detected in said blood sample, the method further comprises a step of administering to said mammal an effective amount of a hemorheologically-active compound.

Circulatory alterations known to occur in exercising horses make such measurements not only applicable, but important for a better understanding of a horse's performance. By utilizing hemorheologic diagnostics one can differentiate normal blood physiologic function from dysfunction, enabling early detection of pathology and therefore, prevent full-blown clinical disease. For example such dysfunctional blood components as echinocytosis, or intensely aggregated red cells, or rigid red cells, or increased red cell concentration, or the potential for generation of microthromboemboli can be detected. These hemorheologic abnormalities would, otherwise, cause increased blood viscosity and would increase flow resistance if the vascular geometry (dilation or constriction) remained unchanged.

As noted above, healthy horses stressed by intense exercise uniquely generate increases from their resting levels in all hemorheological determinants, including red blood cell concentration, red blood cell aggregation, red blood cell rigidity, plasma viscosity, red blood cell shape change (e.g., echinocytes, stomatocytes, and elliptocytes), phosphatidylserine exposure on blood cells, and adhesion molecules. Human athletes exercising at maximal intensity generate small, but significant, increases from their (control) resting levels in all the determinants of blood viscosity; but, the overall blood viscosity difference in horses is significantly greater than that in humans. Therefore, the mechanisms of blood viscosity increase (in normal individuals) differ between the species.

The magnitude of hemorheological abnormalities that occur in healthy horses far outstrips that of any other species even during a disease involving or originating from hemorheopathy. All blood viscosity determinants associated with disease in humans, or those that affect human exercise performance, occur to a much greater extent, naturally and spontaneously, in healthy exercising horses. Horses acquire exercise-induced pulmonary dysfunction, skeletal-muscular injuries, and systemic alterations at a much greater frequency and severity of than do human athletes. The incidence difference in exercise-induced disease and injury between the species apparently occurs due to the more severe hemorheopathy in horses than in humans. Accordingly, horses provide an excellent model for evaluating hemorheologically-active drugs for human application.

Thus, one aspect of the invention is directed to a method of testing a compound to determine if it is hemorheologically active, the method comprising measuring one or more hemorheologic determinants in the blood of said horse prior to administering said compound, administering said compound to the horse, exercising the horse, measuring said one or more hemorheologic determinants in the blood of said horse after exercise, and analyzing the measurements of the one or more hemorheologic determinants, wherein if said compound reduces an increase in said one or more hemorheologic determinants following exercise, it indicates that said compound is hemorheologically active and can be used to treat a hemorheologic abnormality.

### Hemorheology/Hemodynamics Interrelationship

Hemorheology and cardiovascular hemodynamics are distinctly separate, but complementary sciences. Briefly, hemodynamics describes bulk activity of blood in larger vessels (i.e., blood pressure, blood flow, velocity of flow, volume of flow, blood flow conductance, vascular compliance, etc.), whereas, hemorheology, a cellular and molecular approach, describes the interaction (adhesiveness), the flow behavior, the concentration of the individual blood cellular components, and plasma viscosity, factors that influence the blood viscosity and regulate the flow characteristics in the microcirculation.

Both disciplines (hemorheology and hemodynamics) attempt to characterize the resistance to flow in vessels in order to achieve some knowledge of tissue oxygenation. Integrated, the two disciplines (used together) generate research information that can unravel a difficult physiological problem unattainable by either discipline alone. Not only are the two disciplines complementary but, also, they are synergistic. Together, they explain more completely circulatory changes that might induce tissue ischemia. Integral parts of the Hagen-Poiseuille equation of Newtonian fluid viscosity encompass both hemorheologic and hemodynamic measurements. (From: Chien, S. et al. Clinical Hemorheology, p. 27).

Q=r⁴pΔp/8Lη

or

η=r⁴pΔp/8LQ

Where: η=viscosity (a hemorheologic term).
Δp=driving pressure or pressure change (a hemodynamic term).
r⁴=internal radius of tube (both hemorheologic/hemodynamic terms).
   L=length over which Δp occurs (both hemorheologic/hemodynamic terms).
Q=volumetric flow rate (a hemodynamic term).
   p=pi, the constant 3.14159 (ratio of circumference of a circle to its diameter).

These complementary disciplines (hemorheology and hemodynamics), when integrated, help to more fully understand the mechanism of a disease caused by a perturbation of circulatory origin.

While this invention has been particularly shown and described with references to preferred embodiments thereof, it will be understood by those skilled in the art that various changes in form and details may be made therein without departing from the scope of the invention encompassed by the appended claims.

1. A method of treating a hemorheologic abnormality in a non-human mammal, the method comprising administering to said non-human mammal n effective amount of a hemorheologically-active compound.

2. The method of claim 1, further comprising:
(a) obtaining data about one or more hemorheologic determinants in the blood of said non-human mammal before and after administering said hemorheologically-active compound to said non-human mammal; and
(b) analyzing the data about the one or more hemorheologic determinants to assess the effectiveness of administering said hemorheologically-active compound to said non-human mammal.

3. The method of claim 2, wherein said hemorheologic abnormality is selected from the group consisting of an increase in a blood viscosity determinant, an increase in phosphatidlyserine exposure, and an increase in the expression of adhesion molecules on the surface of blood or endothelial cells.

4. The method of claim 3, wherein the blood viscosity determinant comprises red blood cell concentration, red blood cell aggregation, red blood cell rigidity, plasma viscosity, or abnormal red blood cell shape.

5. The method of claim 1, wherein said hemorheologic abnormality is associated with a pulmonary disease or a systemic disease.

6. The method of claim 1, wherein the hemorheologic abnormality is associated with an infectious disease, a toxin, or a venom.

7. The method of claim 1, wherein said hemorheologically-active compound is selected from the group consisting of a plasma membrane stabilizing compound, an anticoagulant compound, a drag reducing compound, an adhesion molecule inhibitor, and a cytokine inhibitor.

8. The method of claim 7, wherein said non-human mammal is a horse, a cow, a sheep, a pig, a dog or a cat.

9. The method of claim 7, wherein said non-human mammal is a horse or a dog and wherein administering said hemorheologically-active compound prevents or reduces an increase in blood viscosity that occurs in the blood of said horse or dog after exercise sufficient to induce increased blood viscosity, the blood of said horse or dog having a normal viscosity prior to said exercise.

10. A method of treating a hemorheologic abnormality in a healthy human, wherein the hemorheologic abnormality occurs in the blood of said healthy human after experiencing a non-disease related stress sufficient to induce the hemorheologic abnormality, the blood of said healthy human having a normal level of a hemorheologic determinant prior to experiencing said non-disease related stress, the method comprising administering to said healthy human an effective amount of a hemorheologically-active compound.

11. The method of claim 10, wherein said non-disease related stress is exercise, exposure to a hot and humid environment, skin burn, exposure to high altitude, underwater diving, hypoxia, surgery, or space travel.

12. The method of claim 10, wherein said hemorheologic abnormality is selected from the group consisting of an increase in a blood viscosity determinant, an increase in phosphatidlyserine exposure, and an increase in the expression of adhesion molecules on the surface of blood or endothelial cells.

13. The method of claim 11, wherein the non-disease related stress is exercise and wherein administering said hemorheologically-active compound prevents or reduces an increase in blood viscosity that occurs in the blood of said healthy human after exercise sufficient to induce increased blood viscosity, the blood of said healthy human having a normal viscosity prior to said exercise.

14. The method of claim 12, wherein said hemorheologically-active compound is selected from the group consisting of a plasma membrane stabilizing compound, an anticoagulant compound, a drag reducing compound, an adhesion molecule inhibitor, and a cytokine inhibitor.

15. A method of evaluating blood flow mechanics in a blood sample from a mammal, the method comprising analyzing said blood sample to determine whether said blood sample comprises a hemorheologic abnormality, wherein, said hemorheologic abnormality is selected from the group consisting of an increase in a blood viscosity determinant, an increase in phosphatidlyserine exposure, and an increase in the expression of adhesion molecules on the surface of blood or endothelial cells, and wherein the presence of said hemorheologic abnormality in said blood sample indicates reduced blood flow mechanics.

16. The method of claim 15, wherein the blood viscosity determinant comprises red blood cell concentration, red blood cell aggregation, red blood cell rigidity, plasma viscosity, or abnormal red blood cell shape.

17. The method of claim 15, further comprising a step of correlating an indication of reduced blood flow mechanics with an increased risk of a pulmonary disease or a systemic disease.

18. The method of claim 15, wherein if said hemorheologic abnormality is detected in said blood sample, the method further comprises a step of administering to said mammal an effective amount of a hemorheologically-active compound.
